(19) United States
(12) Patent Application Publication
(10) Pub. No.: US 2022/0008030 A1
(43) Pub. Date: Jan. 13, 2022

(54) DETECTION OF AGONAL BREATHING USING A SMART DEVICE

(71) Applicant: University of Washington, Seattle, WA (US)

(72) Inventors: Jacob Sunshine, Seattle, WA (US); Justin Chan, Seattle, WA (US); Shyamnath Gollakota, Seattle, WA (US)

(73) Assignee: University of Washington, Seattle, WA (US)

(21) Appl. No.: 17/297,382

(22) PCT Filed: Dec. 20, 2019
(86) PCT No.: PCT/US19/67988

§ 371 Date: May 26, 2021

Related U.S. Application Data
(60) Provisional application No. 62/782,687, filed on Dec. 20, 2018.

(51) Int. Cl. A61B 7/00; G06N 3/08; G06N 3/04; A61B 5/00; A61B 5/08

(52) U.S. Cl. CPC A61B 7/003; G06N 3/08; G06N 3/0454; A61B 2560/0242; A61B 5/0823; A61B 2505/01; A61B 5/4818

(57) ABSTRACT

Examples of systems and methods described herein may classify agonal breathing in audio signals produced by a user using a trained neural network. Examples may include a smart device that may request medical assistance if an agonal breathing event is classified.

### CROSS-REFERENCE TO RELATED APPLICATIONS

This application claims the benefit under 35 U.S.C. § 119 of the earlier filing date of U.S. Provisional Application Ser. No. 62/782,687 filed Dec. 20, 2018, the entire contents of which are hereby incorporated by reference in their entirety for any purpose.

### TECHNICAL FIELD

Examples described herein relate generally to systems for recognizing agonal breathing. Examples of detecting agonal breathing using a trained neural network are described.

### BACKGROUND

Out-of-hospital cardiac arrest (OHCA) is a leading cause of death worldwide and in North America accounts for nearly 300,000 deaths annually. A relatively under-appreciated diagnostic element of cardiac arrest is the presence of a distinctive type of disordered breathing: agonal breathing. Agonal breathing, which arises from a brainstem reflex in the setting of severe hypoxia, appears to be evident in approximately half of cardiac arrest cases reported to 9-1-1. Agonal breathing may be characterized by a relatively short duration of collapse and has been associated with higher survival rates, though agonal breathing may also confuse the rescuer or 9-1-1 operator about the nature of the illness. Sometimes reported as “gasping” breaths, agonal respirations may hold potential as an audible diagnostic biomarker, particularly in unwitnessed cardiac arrests that occur in a private residence, the location of ⅔ of all OHCAs.

Early CPR is a core treatment, underscoring the vital importance of timely detection, followed by initiation of a series of time-dependent coordinated actions which comprise the chain of survival. Hundreds of thousands of people worldwide die annually from unwitnessed cardiac arrest, without any chance of survival because they are unable to activate this chain of survival and receive timely resuscitation. Timely identification and detection of cardiac arrest is important to the ability to provide prompt assistance.

### BRIEF SUMMARY

Example systems are disclosed herein. In an embodiment of the disclosure, an example system includes a microphone configured to receive audio signals, processing circuitry, and at least one computer readable media encoded with instructions which when executed by the processing circuitry cause the system to classify an agonal breathing event in the audio signals using a trained neural network.

Additionally or alternatively, the trained neural network may be trained using audio signals indicative of agonal breathing and audio signals indicative of an ambient noise in an environment proximate the microphone.

Additionally or alternatively, the trained neural network may be trained further using audio signals indicative of non-agonal breathing.

Additionally or alternatively, the non-agonal breathing may include sleep apnea, snoring, wheezing, or combinations thereof.

Additionally or alternatively, the audio signals indicative of non-agonal breathing sounds in the environment proximate to the microphone may be identified from polysomnographic sleep studies.

Additionally or alternatively, the audio signals indicative of agonal breathing may be classified using confirmed cardiac arrest cases from actual agonal breathing events.

Additionally or alternatively, the trained neural network may be configured to distinguish between the agonal breathing event, ambient noise, and non-agonal breathing.

Additionally or alternatively, further included is a communication interface, wherein the instructions may further cause the system to request medical assistance by the communication interface or cause the system to request an AED device be brought to a user.

Additionally or alternatively, the instructions may further cause the system to request confirmation of medical emergency prior to requesting medical assistance by a user interface.

Additionally or alternatively, further included is a display to indicate the request for the confirmation of medical emergency.

Additionally or alternatively, the system may be configured to enter a wake state responsive to the agonal breathing event being classified.

Additionally or alternatively, the instructions may further cause the system to perform audio interference cancellation in the audio signals.

Additionally or alternatively, the instructions may further cause the system to reduce the audio interference transmitted by a smart device housing the microphone.

Example methods are disclosed herein. In an embodiment of the disclosure, an example method includes receiving audio signals, by a microphone, from a user, processing the audio signals by a processing circuitry, and classifying agonal breathing in the audio signals using a trained neural network.

Additionally or alternatively, further included may be training the trained neural network using audio signals indicative of agonal breathing and audio signals indicative of ambient noise in an environment proximate the microphone.

Additionally or alternatively, further included may be cancelling audio interference in the audio signals.

Additionally or alternatively, cancelling the audio interference may further include reducing interfering effects of audio transmissions produced by a smart device including the microphone.

Additionally or alternatively, further included may be requesting medical assistance when a medical emergency is indicated based at least on the audio signals indicative of agonal breathing.

Additionally or alternatively, further included may be requesting confirmation of the medical emergency prior to requesting medical assistance.

Additionally or alternatively, further included may be displaying the request for confirmation of the medical emergency.

### BRIEF DESCRIPTION OF THE DRAWINGS

To easily identify the discussion of any particular element or act, the most significant digit or digits in a reference number refer to the figure number in which that element is first introduced.

FIG. 1 is a schematic illustration of a system arranged in accordance with examples described herein.

FIG. 2 is a schematic illustration of a smart device arranged in accordance with examples described herein.

FIG. 3 is a schematic illustration of the operation of a system arranged in accordance with examples described herein.

FIG. 4 illustrates another example of an agonal breathing pipeline in accordance with one embodiment.

### DETAILED DESCRIPTION

Certain details are set forth herein to provide an understanding of described embodiments of technology. However, other examples may be practiced without various of these particular details. In some instances, well-known circuits, control signals, timing protocols, and/or software operations have not been shown in detail in order to avoid unnecessarily obscuring the described embodiments. Other embodiments may be utilized, and other changes may be made, without departing from the spirit or scope of the subject matter presented here.

Widespread adoption of smart devices, including smartphones and smart speakers, may enable identifying agonal breathing and therefore OHCAs. In some examples, machine learning and algorithms may be used to identify agonal breathing and request medical assistance, such as connecting unwitnessed cardiac arrest victims to Emergency Medical Services (EMS) and cardiopulmonary resuscitation (CPR).

Non-contact, passive detection of agonal breathing allows identification of a portion of previously unreachable victims of cardiac arrest, particularly those who experience such events in a private residence. As the US population ages and more people become at risk for OHCA, leveraging omnipresent smart hardware for monitoring of these emergent conditions can provide public health benefits. Other domains where an efficient agonal breathing classifier could have utility include unmonitored health facilities (e.g., hospital wards and elder care environments), EMS dispatch, and when people have greater than average risk, such as people at risk for opioid overdose-induced cardiac arrest and for people who survive a heart attack.

An advantage of a contactless detection mechanism is that it does not require a victim to be wearing a device while asleep in the bedroom, which can be inconvenient or uncomfortable. Such a solution can be implemented on existing wired smart speakers and as a result would not face power constraints and could scale efficiently. While examples are provided in the context of the victim asleep in the bedroom, the victim may be monitored in other areas such as a bathroom, a kitchen, a living room, a dining room, a hospital room, etc.

Examples described herein may leverage a smart device to present an accessible detection tool for detection of agonal breathing. Examples of systems described herein may operate by (i) receiving audio signals from a user via a microphone of the smart device; (ii) processing the audio signals, and (iii) classifying agonal breathing in the audio signals using a machine learning technique, such as a trained neural network. In some examples, no additional hardware (beyond the smart device) is used. An implemented example system demonstrated high detection accuracy across all interfering sounds while testing across multiple smart device platforms.

For example, a user may produce audio signals indicative of the agonal breathing sounds which are captured by a smart device. The microphone of the smart device may passively detect the user's agonal breathing. While agonal breathing events are relatively uncommon and lack gold-standard measurements, real-world audio of confirmed cardiac arrest cases (e.g., 9-1-1 calls and actual audio from victims experiencing cardiac arrest in a controlled setting such as Intensive Care Unit (ICU), hospice, and planned end of life events) which may include agonal breathing instances captured were used to train a Deep Neural Network (DNN). The trained DNN was used to classify OHCA-associated agonal breathing instances on existing omnipresent smart devices.

Examples of trained neural networks or other systems described herein may be used without necessarily specifying a particular audio signature of agonal breathing. Rather, the trained neural networks may be trained to classify agonal breathing by training on a known set of agonal breathing episodes as well as a set of likely non-agonal breathing interference (e.g., sleep sounds, speech sounds, ambient sounds).

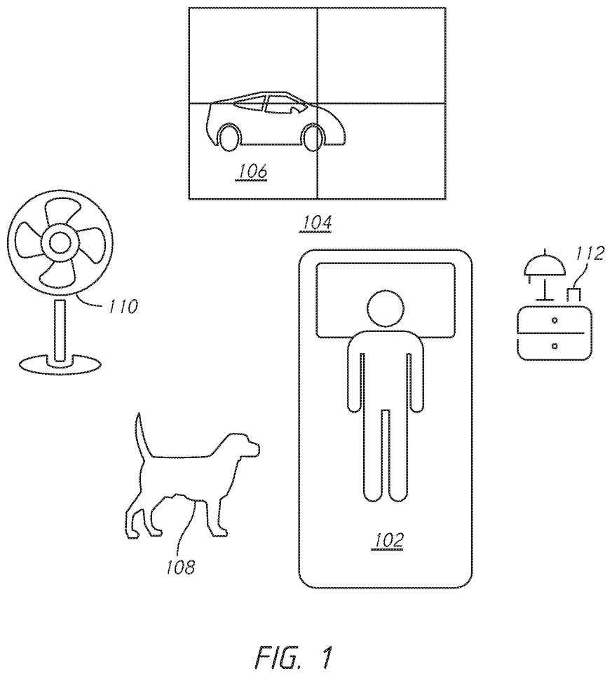

FIG. 1 is a schematic illustration of a system arranged in accordance with examples described herein. The example of FIG. 1 includes user 102, environment 104, traffic noise 106, pet noise 108, ambient noise 110, and smart device 112. The components of FIG. 1 are exemplary only. Additional, fewer, and/or other components may be included in other examples.

Examples of systems and methods described herein may be used to monitor users, such as user 102 of FIG. 1. Generally, a user refers to a human person (e.g., an adult or child). In some examples, neural networks used by devices described herein for classifying agonal breathing may be trained to a particular population of users (e.g., by gender, age, or geographic area), however, in some examples, a particular trained neural network may be sufficient to classify agonal breathing across different populations. While a single user is shown in FIG. 1, multiple users may be monitored by devices and methods described herein.

The user 102 of FIG. 1 is in environment 104. Users described herein are generally found in environments (e.g., settings, locations). The environment 104 of FIG. 1 is a bedroom. While a bedroom setting is shown in FIG. 1, the setting is exemplary only, and devices and systems described herein may be used in other settings. For example, techniques described herein may be utilized in a living room, a kitchen, a dining room, an office, hospital or other medical environments, and/or a bathroom. One building (e.g., house, hospital) may have multiple devices described herein for monitoring agonal breathing in multiple locations in some examples. The user 102 of FIG. 1 is in a bedroom, lying on a bed. In some examples, devices described herein may be used to monitor users during sleep, although users may additionally or instead be monitored in other states (e.g., awake, active, resting).

Generally, environments may contain sources of interfering sounds, such as non-agonal breathing sounds. For example, in FIG. 1, sources of interfering sounds in the environment 104 include pet noise 108, ambient noise 110, and traffic noise 106. Additional, fewer, and/or different interfering sounds may be present in other examples including, but not limited to, appliance or medical device noise or speech. Moreover, the environment 104 may contain non-agonal breathing sounds. In the example of FIG. 1, sleep sounds may be present (e.g., heavy breathing, wheezing, apneic breathing). Systems and devices described herein may be used to classify agonal breathing sounds in the presence of interfering sounds, including non-agonal breathing sounds in some examples. Accordingly, neural network used to classify agonal breathing described herein may be trained using certain common or expected interfering sounds, including non-agonal breathing sounds, such as those discussed with reference to FIG. 1.

Smart devices may be used to classify agonal breathing sounds of a user in examples described herein. In the example of FIG. 1, the smart device 112 may be on a user's nightstand or other location in the environment 104 where the smart device 112 may receive audio signals from the user 102. Smart devices described herein may be implemented using a smart phone (e.g., a cell phone), a smart watch, and/or a smart speaker. The smart device 112 may include an integrated virtual assistant that offers interactive actions and commands with the user 102. Examples of smart phones include, but are not limited to, tablets or cellular phones, e.g., iPhones, Samsung Galaxy phones, and Google Pixel phones. Smart watches may include, but not limited to, Apple Watch, and Samsung Galaxy watch, etc. Smart speakers may include, but not limited to, Google Home, Apple HomePod, and Amazon Echo, etc. Examples of smart device 112 may include a computer, server, laptop, or tablet in some examples. Other examples of smart device 112 may include one or more wearable devices including, but not limited to, a watch, sock, eyewear, necklace, hat, bracelet, ring, or collar. In some examples, the smart device 112 may be of a kind that may be widely available and may therefore easily add to a large number of households an ability to monitor individuals (such as user 102) for agonal breathing episodes. For example, the smart device 112 may include and/or be implemented using an Automated External Defibrillator (AED). In some examples, the AED device may include a display, a microphone, and a speaker and may be used to identify agonal breathing as described herein. In some examples, the smart device 112 may respond to wake words, such as “Hey Siri” or “Hey Alexa.” The smart device 112 may be used in examples described herein to classify agonal breathing. The smart device 112 may not be worn by the user 102 in some examples. Examples of smart devices described herein, such as smart device 112 may utilize a trained neural network to distinguish between (e.g., classify) agonal breathing sounds from noises in the environment 104.

Once agonal breathing sounds are detected by the smart device 112, a variety of actions may be taken. In some examples, the smart device 112 may prompt the user 102 to confirm an emergency is occurring. The smart device 112 may communicate with one or more other users and/or devices responsive to an actual and/or suspected agonal breathing event (e.g., the smart device 112 may make a phone call, send a text, sound or display an alarm, or take other action).

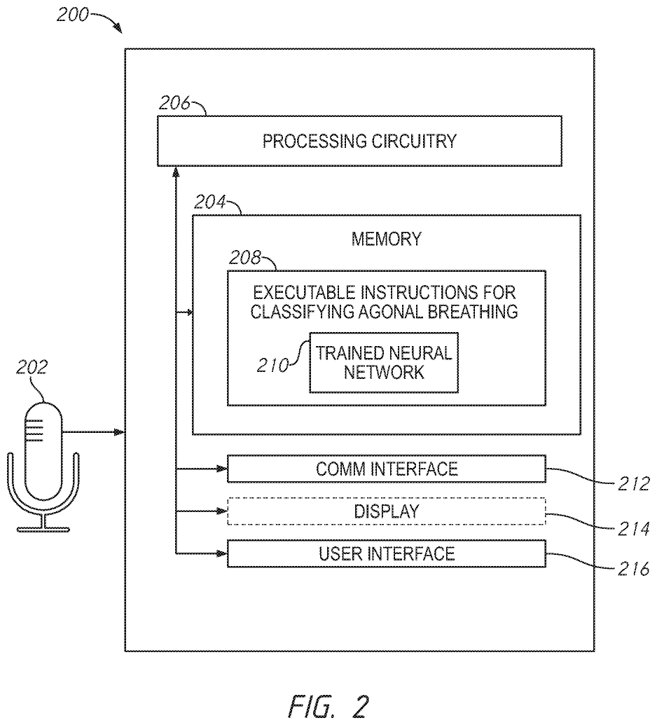

FIG. 2 is a schematic illustration of a smart device arranged in accordance with examples described herein. The system of FIG. 2 includes a smart device 200. The smart device 200 includes a microphone 202 and a processing circuitry 206. The processing circuitry 206 includes a memory 204, communication interface 212, and user interface 216. The memory 204 includes executable instructions for classifying agonal breathing 208 and a trained neural network 210. The processing circuitry 206 may include a display 214. The components shown in FIG. 2 are exemplary. Additional, fewer, and/or different components may be used in other examples. The smart device 200 of FIG. 2 may be used to implement the smart device 112 of FIG. 1, for example.

Examples of smart devices may include processing circuitry, such as processing circuitry 206 of FIG. 2. Any kind or number of processing circuitries may be present, including one or more processors, such as one or more central processing unit(s) (CPUs), graphic processing unit(s) (GPUs), having any number of cores, controllers, microcontrollers, and/or custom circuitry such as one or more application specific integrated circuits (ASICs) and/or field programmable gate arrays (FPGAs).

Examples of smart devices may include memory, such as memory 204 of FIG. 2. Any type or kind of memory may be present (e.g., read only memory (ROM), random access memory (RAM), solid state drive (SSD), secure digital card (SD card)). While a single memory 204 is depicted in FIG. 2, any number of memory devices may be present, and data and/or instructions described may be distributed across multiple memory devices in some examples. The memory 204 may be in communication (e.g., electrically connected) with processing circuitry 206.

The memory 204 may store executable instructions for execution by the processing circuitry 206, such as executable instructions for classifying agonal breathing 208. In this manner, techniques for classifying agonal breathing of a user 102 may be implemented herein wholly or partially in software. Examples described herein may provide systems and techniques which may be utilized to classify agonal breathing notwithstanding interfering signals which may be present.

Examples of systems described herein may utilize trained neural networks. The trained neural network 210 is shown in FIG. 2 and is shown as being stored on memory 204. The trained neural network 210 may, for example, specify weights and/or layers for use in a neural network. Generally, any of a variety of neural networks may be used, including convolutional neural networks or deep neural networks. Generally, a neural network may refer to the use of multiple layers of nodes, where combinations of nodes from a previous layer may be combined in accordance with weights and the combined value provided to one or more nodes in a next layer of the neural network. The neural network may output a classification—for example, the neural network may output a probability that a particular input is representative of a particular output (e.g., agonal breathing).

While a single trained neural network 210 is shown in FIG. 2, any number may be used. In some examples, a trained neural network may be provided specific to a particular population and/or environment. For example, trained neural network 210 may be particular for use in bedrooms in some examples and in classifying as between agonal breathing sounds and non-agonal breathing sleep sounds. During operation, the smart device 200 may provide an indication of an environment in which certain audio sounds are received (e.g., by accessing an association between the microphone 202 and an environment, such as a bedroom), and an appropriate trained neural network may be used to classify sounds from the environment. In some examples, trained neural network 210 may be particular for use in a particular user population, such as adults and/or males. During operation, the smart device 200 may be configured (e.g., a setting may be stored in memory 204) regarding the user and/or population of users intended for use, and the appropriate trained neural network may be used to classify incoming audio signals. In some examples, however, the trained neural network 210 may be suitable for use in classifying agonal breathing across multiple populations and/or environments.

In some examples, the smart device 200 may be used to train the trained neural network 210. However, in some examples the trained neural network 210 may be trained by a different device. For example, the trained neural network 210 may be trained during a training process independent of the smart device 200, and the trained neural network 210 stored on the smart device 200 for use by the smart device 200 in classifying agonal breathing.

Trained neural networks described herein may generally be trained to classify agonal breathing sounds using audio recordings of known agonal breathing events and audio recordings of expected interfering sounds. For example, audio recordings of known agonal breathing events, such as 9-1-1 recordings containing agonal breathing events, may be used to train the trained neural network 210. Other examples of audio recordings of known agonal breathing events (e.g., actual agonal breathing events) may include agonal breathing events occurring in a controlled setting such as a victim in a hospital room, hospice, and experiencing planned end of life, etc. In order to generate a robustly trained neural network, the recordings of known agonal breathing events may be varied in accordance with their expected variations in practice. For example, known agonal breathing audio clips may be recorded at multiple distances from a microphone and/or captured using a variety of smart devices. This may provide a set of known agonal breathing clips from various environments and/or devices. Using such a robust and/or varied data set for training a neural network may promote the accurate classification of agonal breathing events in practice, when an individual may vary in their distance from the microphone and/or the microphone may be incorporated in a variety of devices which may perform differently. In some examples, known non-agonal breathing sounds may further be used to train the trained neural network 210. For examples, audio signals from polysomnographic sleep studies may be used to train trained neural network 210. The non-agonal breathing sounds may similarly be varied by recording them at various distances from a microphone, using different devices, and/or in different environments. The trained neural network 210 trained on recordings of actual agonal breathing events, such as 9-1-1 recordings of agonal breathing and expected interfering sounds such as polysomnographic sleep studies may be particularly useful, for example, for classifying agonal breathing events in a bedroom during sleep.

Examples of smart devices described herein may include a communication interface, such as communication interface 212. The communication interface 212 may include, for example, a cellular telephone connection, a Wi-Fi connection, an Internet or other network connection, and/or one or more speakers. The communication interface 212 may accordingly provide one or more outputs responsive to classification of agonal breathing. For example, the communication interface 212 may provide information to one or more other devices responsive to a classification of agonal breathing. In some examples, the communication interface 212 may be used to transmit some or all of the audio signals received by the smart device 200 so that the signals may be processed by a different computing device to classify agonal breathing in accordance with techniques described herein. However, in some examples to aid in speedy classification and preserve privacy, audio signals may be processed locally to classify agonal breathing, and actions may be taken responsive to the classification.

Examples of smart devices described herein may include one or more displays, such as display 214. The display 214 may be implemented using, for example, one or more LCD displays, one or more lights, or one or more touchscreens. The display 214 may be used, for example, to display an indication that agonal breathing has been classified in accordance with executable instructions for classifying agonal breathing 208. In some examples, a user may touch the display 214 to acknowledge, confirm, and/or deny the occurrence of agonal breathing responsive to a classification of agonal breathing.

Examples of smart devices described herein may include one or more microphones, such as microphone 202 of FIG. 2. The microphone 202 may be used to receive audio signals in an environment, such as agonal breathing sounds and/or interfering sounds. While a single microphone 202 is shown in FIG. 2, any number may be provided. In some examples, multiple microphones may be provided in an environment and/or location (e.g., building) and may be in communication with the smart device 200 (e.g., using wired and/or wireless connections, such as Bluetooth, or Wi-Fi). In this manner, a smart device 200 may be used to classify agonal breathing from sounds received through multiple microphones in multiple locations.

In some examples, smart devices described herein may include executable instructions for waking the smart device. Executable instructions for waking the smart device may be stored, for example, on memory 204. The executable instructions for waking the smart device may cause certain components of the smart device 200 to turn on, power up, and/or process signals. For example, smart speakers may include executable instructions for waking responsive to a wake word, and may process incoming speech signals only after recognizing the wake word. This waking process may cut down on power consumption and delay during use of the smart device 200. In some examples described herein, agonal breathing may be used as a wake word for a smart device. Accordingly, the smart device 200 may wake responsive to detection of agonal breathing and/or suspected agonal breathing. Following classification of agonal breathing, one or more components of the device may power on and/or conduct further processing using the trained neural network 210 to confirm and further classify an agonal breathing event and take action responsive to the agonal breathing classification.

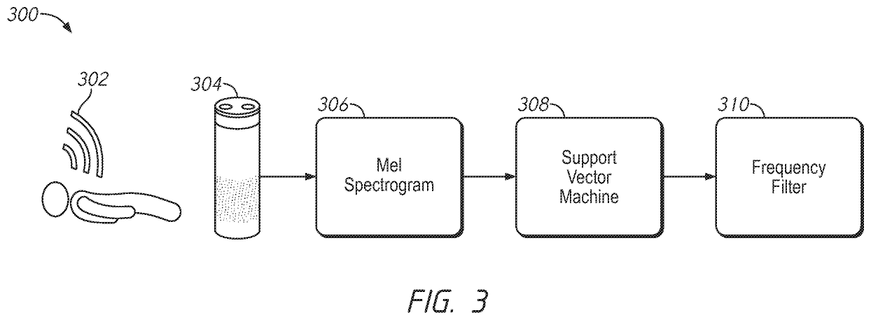

FIG. 3 is a schematic illustration of the operation of a system arranged in accordance with examples described herein. FIG. 3 depicts user 302, smart device 304, spectrogram 306, Support vector machine 308, and frequency filter 310. The user 302 may be, for example, the user 102 in some examples. The smart device 304 may be the smart device 112, for example. The components and/or actions shown in FIG. 3 are exemplary only, and additional, fewer, and/or different components may be used in other examples.

In the example of FIG. 3, the user 302 may produce agonal breathing sounds. The smart device 304 may include a trained neural network, such as the trained neural network 210 of FIG. 2. The trained neural network may be, for example, a convolutional neural network (CNN). During operation, the smart device 304 may receive audio signals produced by the user 302 and may provide them to a trained neural network for classifying agonal breathing, such as the trained neural network 210 of FIG. 2.

The neural network may be trained to output probabilities (e.g., a stream of probabilities in real-time) indicative of a likelihood of agonal breathing at a particular time. The incoming audio signals may be segmented into segments which are of a duration relevant to agonal breathing. For example, audio signals occurring during a particular time period expected to be sufficient to capture an agonal breath may be used as segments and input to the trained neural network to classify or begin to classify agonal breathing. In some examples, a duration of 2.5 seconds may be sufficient for reliably capturing an agonal breath. In other examples, a duration of 1.5 seconds, 1.8 seconds, 2.0 seconds, 2.8 seconds, 3.0 seconds may be sufficient.

Each segment may be transformed from the time-domain into the frequency domain, such as into a spectrogram, such as a log-mel spectrogram 306. The transformation may occur, for example, using one or more transforms (e.g., Fourier transform) and may be implemented using, for example, the processing circuitry 206 of FIG. 2. The spectrogram may represent a power spectral density of the signal, including the power of multiple frequencies in the audio segment as a function of time. In some examples, each segment may be further compressed into a feature embedding using a feature extraction and/or feature embedding technique, such as principal component analysis. The feature embedding may be provided to a neural network, such as Support vector machine 308 (SVM). In some examples, the Support vector machine 308 may have a radial basis function kernel that can distinguish between agonal breathing instances (e.g., positive data) and non-agonal breathing instances (e.g., negative data). An agonal breathing frequency filter 310 may then be applied to the classifier's probability outputs to reduce the false positive rate of the overall system. The frequency filter 310 may check if the rate of positive predictions is within the typical frequency at which agonal breathing occurs (e.g., within a range of 3-6 agonal breaths per minute).

In some examples, in addition to agonal breathing sounds, the user 302 may produce sleep sounds such as movement in bed, breathing, snoring, and/or apnea events. While apnea events may sound similar to agonal breathing, they are physiologically different from agonal breathing. Examples of trained neural networks described herein, including trained neural network 210 of FIG. 2 and Support vector machine 308 of FIG. 3, may be trained to distinguish between agonal breathing and non-agonal breathing sounds (e.g., apnea events). In some examples, the smart device 304 may use acoustic interference cancellation to reduce the interfering effects of its own audio transmission and improve detection accuracy of agonal breathing. For example, the processing circuitry 206 and/or executable instructions shown in FIG. 2 may include circuitry and/or instructions for acoustic interference calculation. The audio signals generated by the user 302 may have cancellation applied, and the revised signals may be used as input to a trained neural network, such as trained neural network 210 of FIG. 2.

Neural networks described herein, such as the trained neural network 210 and/or Support vector machine 308 of FIG. 3 may be trained using positive data (e.g., known agonal breathing audio clips) and negative data (e.g., known interfering noise audio clips). In one example, the trained neural network 210 was trained on negative data spanning over 600 audio event classes. Negative data may include non-agonal audio event categories which may be present in the user 302's surroundings: snoring, ambient noise, human speech, sounds from a television or radio, cat or dog sounds, fan or air conditioner sounds, coughing, and normal breathing, for example. A k-fold (e.g., k=10) cross-validation may be applied to the model for detecting unwitnessed agonal breathing. Further in training neural networks, receiver-operating characteristic (ROC) curves may be generated to compare the performance of the classifier against other sourced negative classes. The ROC curve for a given class may be generated using k-fold validation. The validation set in each fold may be set to contain negative recordings from only a single class in some examples to promote and/or ensure class balance between positive and negative data.

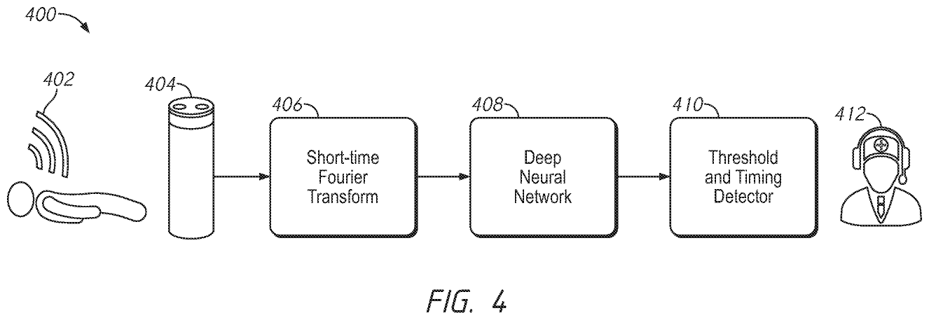

FIG. 4 is a schematic illustration of a system arranged in accordance with examples described herein. The example of FIG. 4 includes user 402, smart device 404, short-time Fourier transform 406, deep neural network 408, and threshold and timing detector 410. The short-time Fourier transform 406, deep neural network 408, and threshold and timing detector 410 are shown schematically separate from the smart device 404 to illustrate a manner of operation, but may be implemented by the smart device 404. The smart device 404 may be used to implement and/or may be implemented by, for example, the smart device 112 of FIG. 1, smart device 200 of FIG. 2, and/or smart device 304 of FIG. 3. The deep neural network 408 may be used to implement and/or may be implemented by trained neural network 210 of FIG. 2 and/or Support vector machine 308 of FIG. 3. The components shown in FIG. 4 are exemplary only. Additional, fewer, and/or different components may be used in other examples.

In the example of FIG. 4, the user 402 may produce breathing noises, which may be picked up by the smart device 404 as audio signals. The audio signals received by the smart device 404 may be converted into a spectrogram using, for example a Fourier transform, e.g., short-time Fourier transform 406. In some examples, a 448-point Fast Fourier Transform Hamming may be used. The short-time Fourier transform 406 may be implemented, for example, using processing circuitry 206 and/or executable instructions executed by processing circuitry 206 of FIG. 2. In some examples, the window size may be 188 samples, of which 100 samples overlap between time segments. A spectrogram may result. In some examples, the spectrogram may be generated, for example by providing power values in decibels and mapping the power values to a color (e.g., using the jet colormap Matlab). In some examples, a maximum and minimum power spectral density were −150 and 50 db/Hz respectively, although other values may be used and/or encountered. The spectrogram may be resized to a particular size for use as input to a neural network, such as deep neural network 408. In some examples, a 224 by 224 image may be used for compatibility with the deep neural network 408, although other sizes may be used in other examples. When the deep neural network 408 determines the user 402 is making agonal breathing sounds, the smart device 404 may be triggered to take action, such as to seek medical help from EMS 412 or other medical providers registered with the smart device 404.

On average, instances of agonal breathing may be separated by a period of negative sounds (e.g., interfering sounds). In some examples, the period of time separating instances of agonal breathing sounds may be 30 seconds, although other periods may be used in other examples. The threshold and timing detector 410 may be used to detect agonal breathing sounds and reduce false positives by only classifying agonal breathing as an output when agonal breathing sounds are classified over a threshold number of times and/or within a threshold amount of time. For example, in some examples agonal breathing may only be classified as an output if it is classified by a neural network more than one time within a time frame, more than two times within a time frame, or more than another threshold of times. Examples of time frames may be 15 seconds, 20 seconds, 25 seconds, 30 seconds, 35 seconds, 40 seconds, and 45 seconds.

When it is determined that the user 402 is producing agonal breathing, the smart device 404 may contact EMS 412, caregivers, or volunteer responders in the neighborhood to assist in performing CPR and/or any other necessary medical assistance. Additionally or alternatively, the smart device 404 may prompt the EMS 412, caregivers, or volunteer responders to bring an AED device be brought to a user. The AED device may provide visual and/or audio prompts for operating the AED device and performing CPR.

In an example, the smart device 404 may reduce and/or prevent false alarms of requesting medical help from EMS 412 when the user 402 does not in fact have agonal breathing by sending a warning to the user 402 (e.g., by displaying an indication that agonal breathing has been classified and/or prompting a user to confirm an emergency is occurring). The smart device 404 may send a warning and seek an input other than agonal breathing sounds from the user 402 via the user interface 216. The warning may additionally be displayed on display 214. Absent a confirmation from the user 402 that the agonal breathing sounds detected is not indicative of agonal breathing, the communication interface 212 of smart device 404 may seek medical assistance in some examples. In some examples, an action (e.g., seeking medical assistance) may only be taken responsive to confirmation an emergency is occurring.

Utilizing smart devices may improve the ubiquity with which individuals may be monitored for agonal breathing events. By prompt and passive detection of agonal breathing, individuals suffering cardiac arrest may be able to be treated more promptly, ultimately improving outcomes and saving lives.

### Implemented Example

An implemented example system was used to train and validate a model for detecting unwitnessed agonal breathing of real-world sleep data. In training the neural network, agonal breathing recordings sourced from 9-1-1 emergency calls from 2009 to 2017, provided by Public Health Seattle & King County, Division of Emergency Medical Services. The positive dataset included 162 calls (19 hours) that had clear recordings of agonal breathing. For each occurrence, 2.5 seconds of audio from the start of each agonal breathing was extracted. A total of 236 clips of agonal breathing instances were extracted. The agonal breathing dataset was augmented by playing the recordings over the air over distances of 1, 3, and 6 m, in the presence of interference from indoor and outdoor sounds with different volumes and when a noise cancellation filter is applied. The recordings were captured on different devices, namely an Amazon Alexa, an iPhone 5s and a Samsung Galaxy S4 to get 7316 positive samples.

The negative dataset included 83 hours of audio data captured during polysomnographic sleep studies, across 12 different patients. These audio streams include instances of hypopnea, central apnea, obstructive apnea, snoring, and breathing. The negative dataset also included interfering sounds that might be present in a bedroom while a person is asleep, specifically a podcast, sleep soundscape and white noise. In training the model, 1 hour of audio data from the sleep study in addition to other interfering sounds were used. These audio signals were played over the air at different distances and recorded on different devices to get 7305 samples. The remaining 82 hours of sleep data (117,985 audio segments) is then used for validating the performance of the model.

A k-fold (k=10) cross-validation was applied and an area under the curve (AUC) of 0.9993±0.0003 was obtained. An operating point with an overall sensitivity and specificity of 97.24% (95% CI: 96.86-97.61%) and 99.51% (95% CI: 99.35-99.67%), respectively, was obtained. The k-fold (k=10) cross-validation using other machine learning classifiers including k-nearest neighbors, logistic regression and random forests was executed. These classifiers achieved an AUC that was >0.98 but slightly lower than the AUC of the trained SVM. The detection algorithm can run in real-time on a smartphone natively and can classify each 2.5 s audio segment within 21 ms. With a smart speaker, the algorithm can run within 58 ms. The audio embeddings of the dataset were visualized by using t-SNE to project the features into a 2-D space.

To evaluate false positive rate, the classifier trained over the full audio stream collected in the sleep lab was run. The sleep audio used to train each model was excluded from evaluation. By relying only on the classifier's probability outputs, a false positive rate of 0.14409% was obtained (170 of 117,985 audio segments). To reduce false positives, the classifier's predictions are passed through a frequency filter that checks if the rate of positive predictions is within the typical frequency at which agonal breathing occurs (e.g., within a range of 3-6 agonal breaths per minute). This filter reduced the false positive rate to 0.00085%, when it considers two agonal breaths within a duration of 10-20 s. When it considers a third agonal breath within a subsequent period of 10-20 s, the false positive rate reduces to 0%.

Outside of the sleep lab, real-world recordings of sleep sounds that occur within the house (e.g., snoring, breathing, movement in bed) were used to evaluate the false positive rate of the classifier. 35 individuals were recruited to record themselves while sleeping using their smart devices for a total duration of 167 hours. The recordings were manually checked to ensure the audio corresponded to sleep sounds. The classifier was retrained with an additional 5 min of data from each subject, with a comparable operating point with a sensitivity and specificity of 97.17% (95% CI: 96.79-97.55%) and 99.38% (95% CI: 99.20-99.56%), respectively. The false positive rate of the classifier without a frequency filter is 0.21761%, corresponding to 515 of the 236,666 audio segments (164 hours) used as test data. After applying the frequency filter, the false positive rate reached 0.00127% when considering two agonal breaths within a duration of 10-20 seconds, and 0% after considering a third agonal breath within a subsequent period of 10-20 seconds.

Audio clips of agonal breathing over the air from an external speaker and captured the audio on an Amazon Echo and Apple iPhone 5s. The detection accuracy was evaluated using the k=10 validation folds in the dataset such that no audio file in the validation set appears in any of the different recording conditions in the training set. Both the Echo and iPhone 5s achieved >96.63% mean accuracy at distances up to 3 meters. When the smart device was placed in a pocket, with the user supine on the ground and the speaker next to the head, a mean detection accuracy of 93.22±4.92%. Across all interfering sound classes including indoor interfering sounds (e.g., cat, dog, air conditioner) and outdoor interfering sounds (e.g., traffic, construction and human speech), the smart device achieved a mean detection accuracy of 96.23%.

A smart device was set to play sounds one might play to fall asleep (e.g., a podcast, sleep soundscape, and white noise). These sounds were played at a soft (45 dbA) and loud (67 dBA) volume. Simultaneously, the agonal breathing audio clips were played. When the audio cancellation algorithm was applied, the detection accuracy achieved an average of 98.62 and 98.57% across distances and sounds for soft and loud interfering volumes, respectively.

To benchmark the classifier's performance against negative audio sounds, a stream of negative sounds was streamed over the air: snoring, a podcast, a sleep soundscape and white noise, and the negative audio sounds were recorded on a smart device. The smart device achieved a mean detection accuracy of 99.57% at a distance of 3 m; a 100% accuracy corresponds to the classifier correctly identifying that the sounds are from the negative dataset. Across all interfering sounds, the mean detection accuracy was 99.29%.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

1. A system comprising:
a microphone is configured to receive audio signals;
a processing circuitry; and
at least one computer readable media encoded with instructions which when executed by the processing circuitry cause the system to classify an agonal breathing event in the audio signals using a trained neural network.

2. The system of claim 1, wherein the trained neural network is trained using audio signals indicative of agonal breathing and audio signals indicative of an ambient noise in an environment proximate the microphone.

3. The system of claim 2, wherein the trained neural network is trained further using audio signals indicative of non-agonal breathing.

4. The system of claim 3, wherein the non-agonal breathing comprises sleep apnea, snoring, wheezing, or combinations thereof.

5. The system of claim 2, wherein the audio signals indicative of non-agonal breathing sounds in the environment proximate to the microphone are identified from polysomnographic sleep studies.

6. The system of claim 2, wherein the audio signals indicative of agonal breathing are classified using confirmed cardiac arrest cases from actual agonal breathing events.

7. The system of claim 1, wherein the trained neural network is configured to distinguish between the agonal breathing event, ambient noise, and non-agonal breathing.

8. The system of claim 1, further comprising a communication interface, wherein the instructions further cause the system to request medical assistance by the communication interface or cause the system to request an AED device be brought to a user.

9. The system of claim 8, wherein the instructions further cause the system to request confirmation of medical emergency prior to requesting medical assistance by a user interface.

10. The system of claim 9, further comprising a display to indicate the request for the confirmation of medical emergency.

11. The system of claim 1, wherein the system is configured to enter a wake state responsive to the agonal breathing event being classified.

12. The system of claim 1, wherein the instructions further cause the system to perform audio interference cancellation in the audio signals.

13. The system of claim 12, wherein the instructions further cause the system to reduce the audio interference transmitted by a smart device housing the microphone.

14. A method comprising:
receiving audio signals, by a microphone, from a user;
processing the audio signals by a processing circuitry; and
classifying agonal breathing in the audio signals using a trained neural network.

15. The method of claim 14, further comprising training the trained neural network using audio signals indicative of agonal breathing and audio signals indicative of ambient noise in an environment proximate the microphone.

16. The method of claim 14, further comprising cancelling audio interference in the audio signals.

17. The method of claim 16, wherein cancelling the audio interference further comprises reducing interfering effects of audio transmissions produced by a smart device comprising the microphone.

18. The method of claim 14, further comprising requesting medical assistance when a medical emergency is indicated based at least on the audio signals indicative of agonal breathing.

19. The method of claim 18, further comprising requesting confirmation of the medical emergency prior to requesting medical assistance.

20. The method of claim 19, further comprising displaying the request for confirmation of the medical emergency.
